(19) United States
(12) Patent Application Publication
(10) Pub. No.: US 2011/0054054 A1
(43) Pub. Date: Mar. 3, 2011

(54) HYDRAZINE-MODIFIED MATRICES AND USE THEREOF IN METHODS FOR ISOLATING BIOMOLECULES

(75) Inventors: Roland Fabis, Leverkusen (DE); Ralf Himmelreich, Langenfeld (DE)

(73) Assignee: QIAGEN GMBH, Hilden (DE)

(21) Appl. No.: 12/866,857

(22) PCT Filed: Feb. 20, 2009
(86) PCT No.: PCT/EP2009/052051

§ 371 Date: Nov. 19, 2010

(30) Foreign Application Priority Data
Feb. 22, 2008 (DE) 10 2008 010 692.5

(51) Int. Cl. C07H 1/06; C08J 9/00; C07H 21/00

(52) U.S. Cl. 521/149; 536/25.4; 536/22.1

(57) ABSTRACT

The invention relates to novel functionalized matrices or matrix materials comprising structures of general formula Carrier-Linker-Spacer-NR₃—NR₁R₂ (I), methods for the production thereof, and the use thereof for isolating biomolecules.

Träger—Linker—Spacer—NR₃—NR₁R₂

The present invention relates to functionalized matrices and matrix materials which comprise structures of the general formula I

carrier-linker-spacer-NR₃—NR₁R₂ (I),

methods for production thereof and also the use thereof for isolating biomolecules.

Biomolecules according to the present invention are taken to mean quite generally molecules which, as products of an evolutionary selection, fulfill specific tasks for an organism by means of ordered and selective interaction and make up the basis of its vital functions [cf. Römpp, Lexikon Biochemie and Molekularbiologie [Römpp's Lexicon of Biochemistry and Molecular Biology], Georg Thieme Verlag, Stuttgart 1999].

In particular, in the context of the present invention, nucleic acids such as, e.g., plasmid DNA, chromosomal DNA, total RNA, mRNA or RNA/DNA hybrids are considered to be biomolecules.

The method according to the invention relates to the purification and isolation of these biomolecules from aqueous systems which contain, e.g., impurities such as fats or cell debris.

In addition, the present invention relates to the isolation or purification of optionally artificial biomolecules from reaction mixtures which result from laboratory or industrial synthesis reactions.

The invention relates in particular to matrices which have the ability to immobilize nucleic acids at a pH in a first interval and to release them again at a pH in a second interval which differs from the first pH or pH interval. The invention additionally relates to methods for production thereof use thereof for purifying or isolating the desired nucleic acid(s).

The great scientific progress in the field of molecular biology—inter alia in the field of genetic engineering and molecular diagnostics—has given rise to a great requirement for rapid, reliable and automatable methods for isolating and purifying nucleic acids.

The biological samples which are used, inter alia, for DNA identification or analysis can originate from a multiplicity of sources such as, e.g. animal and plant cells, feces, tissue and bone samples (biopsies) or blood. In addition, the samples can also be obtained from soil, from foods or from synthetically produced nucleic acid mixtures.

Many methods of molecular biology such as reverse transcription, cloning restriction analysis, amplification and also sequencing demand necessarily that the nucleic acids used in the respective methods are essentially free of impurities which could adversely affect the corresponding reaction sequences or analytical methods. Such impurities generally comprise substances which catalyze or initiate the breakdown or depolymerization of a nucleic acid, or substances which block the detection of the nucleic acid or mask the nucleic acid. Unwanted impurities comprise, in addition, macromolecular substances such as, e.g., enzymes, proteins, polysaccharides or polynucleotides, and also substances having a low molecular weight, such as lipids, enzyme inhibitors or oligonucleotides.

Further impurities are dyes (pigments), trace elements (metals) or organic solvents.

These requirements have led to the fact that over the course of years, in the prior art, a multiplicity of purification and isolation methods have already become established. For instance, DNA, for example, can be obtained by salting out in the presence of phenol and trichloromethane. On the other hand, DNA and RNA can be obtained by using chaotropic salts on mineral carriers (such as, e.g. silica) or—derivatized—silica resins. The use of magnetic particles—what are termed “magnetic beads”—has become established with respect to use in automated methods.

Marko et al. [Analyt. Biochem. 121 (1982) 382] and also Vogelstein et al. [Proc. Nat. Acad. Sci. 76 (1979) 615], first recognized that if the DNA in nucleic acid-containing extracts is exposed to high concentrations of sodium iodide or sodium perchlorate, only the DNA binds to mechanically finely comminuted glass scintillation tubes and also comminuted glass fiber membranes or glass fiber plates, while RNA and proteins do not bind. The DNA thus bound can in the simplest case be eluted with water. In the ensuing period, accordingly, numerous patents have been published which are based on this type of DNA isolation.

Thus, the European laid-open patent EP-A-0 389 063 relates to a method for isolating nucleic acids from a biological source. According to this method, the nucleic acid-containing biological sources such as blood, cells, plasma etc. are disrupted in the presence of high concentrations of chaotropic salts. The nucleic acids are then bound to a silica surface. The nucleic acids are thereafter washed and eluted.

U.S. Pat. No. 5,155,018 describes a method for isolating RNA from biological sources which, in addition to RNA, also contain DNA and also other components. In this method, the biological sample is acidified and mixed with a chaotropic agent such as, e.g., a guanidinium salt. Silicate particles are added to the sample. Under the given conditions, RNA binds to the silicate particles.

In this method, also the RNA is then separated off from the particles.

Colpan et al. describe, in the international patent application WO-A-95/01359, a method for purifying and separating nucleic acid mixtures by adsorption of the nucleic acid from an alcoholic solution of high ionic strength.

The adsorption solution, in addition to alcohol at a concentration of 1 to 50 vol %, contains salts in a concentration of 1 to 10 M, wherein chaotropic salts such as, e.g., guanidinium thiocyanate, sodium perchlorate or guanidinium hydrochloride are preferred.

WO-A-95/21849 further relates to a method for separating double- and/or single-stranded nucleic acids from sources which contain these nucleic acids. In this method also, the nucleic acids are adsorbed to mineral carriers under conditions which make possible binding of the desired nucleic acid species, whereas the unwanted nucleic acid species does not bind to these mineral carriers.

In order to bind predominantly single-stranded nucleic acids to a mineral carrier and thereby separate them from double-stranded nucleic acids, the treatment conditions for the samples containing both nucleic acid species are set accordingly with an aqueous mixture of salts, in particular chaotropic salts and alcohol. The non-adsorbed double-stranded nucleic acid can then be further purified or isolated using known methods.

Further methods are disclosed, for example, in the European laid-open patents EP-A-512 767 and EP-A-515 484, in the international patent applications WO-A-95/13368, WO-A-96/18731 and WO-A-97/10331 and also in U.S. Pat. No. 4,923,978 and U.S. Pat. No. 5,057,426.

European laid-open patent EP-A-0 707 077 describes the binding and also subsequent selective release of nucleic acid from a lyzate by means of a water-soluble weakly basically reacting polymer which forms a precipitate with the nucleic acid under specific conditions. This precipitate can be separated off from the aqueous solution and the nucleic acid can be released employing high salt concentrations and also with the action of strong bases or by heating.

With respect to all of the above-described isolation and purification methods, with regard to the biological samples it must be stated that blood is a source for DNA analyses which is available in the largest amounts, since blood samples are routinely taken for a wide variety of reasons. Owing to its viscous nature, and also due to containing large amounts of protein, the automation of isolating nucleic acids from this feedstock gave rise again and again to unexpected difficulties—a circumstance which is also documented repeatedly in the prior art. In addition, it was found—with respect to automation—that handling large sample volumes which have relatively low DNA contents was difficult.

Therefore, the object of the present patent application was to supply at least in part a solution to the above-described problems known from the prior art. The present invention therefore targets in the most general sense the requirement for materials and methods which make possible a rapid and efficient method for isolating nucleic acids from a mixture of the desired nucleic acids with impurities.

This object is achieved in one embodiment according to the invention by functionalized matrices of the general formula I and also by methods for isolating one or more nucleic acids such as, e.g., plasmid DNA, chromosomal DNA, total RNA, mRNA, or RNA/DNA hybrids, e.g. from biological samples which contain impurities such as proteins, fats, cell debris etc.

In a further embodiment according to the invention, the present invention relates to matrix materials or matrices modified with hydrazine or with a hydrazine derivative in which the hydrazine partial structures are bound to the carrier via what is termed a linker, and also to methods for producing the matrices of the general formula (I).

According to a further aspect, the invention likewise relates to a method for isolating biomolecules—in particular nucleic acids—using the abovementioned matrix modified with hydrazine or with a hydrazine derivative, which method comprises the following steps:

(a) providing the matrix materials or matrices modified with hydrazine or with a hydrazine derivative;

(b) combining the matrix with a mixture containing the biomolecule to be isolated or containing preferably the nucleic acid to be isolated in addition to at least one impurity;

(c) incubating the matrix and the mixture at an adsorption pH, wherein the biomolecule or preferably the desired nucleic acid is immobilized on the matrix;

(d) separating the matrix containing the biomolecule or containing the immobilized nucleic acid(s) from the mixture;

(e) combining the matrix containing the biomolecule or containing the immobilized nucleic acid(s) with an elution solution at a desorption pH, wherein the desired biomolecule(s) or nucleic acid(s) is/are desorbed from the matrix.

The carrier or the carrier surface or the underlying carrier material can be formed from any customary—polymeric—inorganic or organic material, including soft gel-type carriers such as agarose, polyacrylamide or cellulose or hard carrier material, such as polystyrene, latex methacrylate or of a metal oxide such as preferably silica. If the carrier material is embodied by silicon dioxide, it is preferably in the form of silica gel, solid glass or diatomaceous earth, or in the form of a mixture of these substances. In the context of the present invention, a polymethacrylate is particularly preferred as homopolymer, but also as copolymer with further styrene monomers, acrylates or methacrylates, and also crosslinkers, such as divinylbenzene or ethylene glycol dimethacrylate.

When organic polymeric materials are used, any polymer can be selected which can be functionalized in such a manner that the functional groups (linkers) which may be already present in the polymer as precursor or be further generated within a subsequent reaction—can react with hydrazine or with the desired hydrazine derivative with formation of a covalent bond.

In a further embodiment, the linkers can optionally react with a spacer—preferably with formation of a covalent bond—wherein the hydrazine or the hydrazine derivative is already bound to the linker or is not bound to the linker until after a further reaction. The linkers can according to the invention also react directly with hydrazine itself or with the desired hydrazine compound.

Carrier in the context of the present invention means essentially a polymeric backbone which is made up by the polymerization of the starting monomers optionally with a crosslinker and optionally with other auxiliaries. Auxiliaries are taken to mean according to the invention primarily substances which affect, for example, the chemical or physical properties of the polymer. These include, for example, pore-forming agents which affect the porosity of the polymer or substances which give the polymer magnetic properties. The carrier can therefore—if desired—be bound to a material which has a permanent or temporary magnetic moment.

The geometric shape in which the polymer—which forms the matrix—is present is of very little significance in this case. For instance, the surfaces can be generated, for example, in Eppendorff tubes or Falcon tubes, microtiter plates, PCR plates or, for example, on microscope slides.

The matrix can also be formed as what is termed a composite in which the matrix material of the general formula I can be present, for example, in combination with an inorganic polymer—such as, e.g. silica (SiO₂)— or applied to a metallic surface—such as, e.g. gold or iron—or can be combined in any other form with other materials.

Very generally, it is possible for producing the matrix to apply the functionalized polymers (matrix materials) to any hydrophilic or hydrophobic—optionally precoated—surface, which results in a broad spectrum of applications (inter alia in multiwell systems such as, e.g. microtiter plates or microfluidic systems).

In the context of the invention the carrier of the matrix is formed by a polymer, wherein the expression “polymer” is understood in its broadest definition. According to the invention polymers in the context of the present invention are embodied by addition polymers, polycondensates and polyadducts. According to the present invention, the carrier can be made up of homopolymers, copolymers, terpolymers etc. or of mixtures of different polymeric components. The optionally different polymers can—if this is desired or appears to be expedient—be crosslinked among one another by what is termed a crosslinker.

Both carrier materials—organic or else inorganic carrier material—can be present inter alia in the form of particles, solid parts—such as, for example, vessel walls of reaction vessels—membranes or in the form of nonwovens or woven fabrics.

A further possibility is to bind hydrazine—or a corresponding derivative of hydrazine—to a (water-) soluble polymer as is disclosed, for example, in the European patent application EP-A-0 707 077 already cited at the outset.

The surfaces of the matrices modified with hydrazine or with one or more derivatives thereof therefore show in principle the following structure, wherein—as already mentioned—the spacer can optionally be omitted, i.e. it embodies merely a single bond:

carrier-linker-spacer-NR₃—NR₁R₂ (I),

R₃ can in this case also, together with the spacer or optionally directly with the linker, form a further bond or a double bond

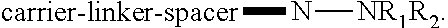

Therein, the carrier surface is a carrier of an organic or inorganic material—preferably an inorganic or organic polymer—which can optionally further comprise other functional groups.

In the simplest case, the linker embodies a single bond, but can, depending on the chosen starting functionality, an up to six-membered alkylene bridge which, in addition to methylene groups—optionally substituted with halogen, hydroxyl, amino and thiol groups—contains partial structures selected from the group of the following structural elements comprising —O—, —NH—, —S—, —C(═O)—, —C(═O)—C(═O)—, —C(═O)—O—, —C(═O)—C(═O)—O—, —C(═S)—O—, —C(═O)—S—, —C(═O)—N—, —NH—C(═O)—NH—.

The spacer, in the simplest case, is a single or double bond, an alkylene bridge containing 1 to 12 carbon atoms which can optionally be interrupted by oxygen or sulfur atoms or else amino groups, and which optionally can comprise one or more carbonyl or carboxyl groups. Examples which may be mentioned are dialdehyde, polyaldehyde, dicarboxyl or polycarboxyl partial structures. In addition, ethylene glycol diglycidyl ether, glycerol diglycidyl ether, glycerol triglycidyl ether structures in addition to ethylene glycol, polyethylene glycol, propylene glycol and polypropylene glycol and also derivatives thereof may be mentioned as spacers.

To the optionally present spacer is bound, via a covalent single bond or via a double bond, the hydrazine partial structure in which the substituents R₁, R₂ and optionally R₃ independently of one another can be hydrogen, C₁-C₆ alkyl, C₃-C₆ alkenyl, C₃-C₆ alkynyl, a higher alkyl group, a cycloalkyl group or an aryl or aralkyl group.

According to the invention, the abovementioned substituents—unless stated otherwise—have the following meanings in the context of the present invention:

C₁-C₆ alkyl—generally also termed only “alkyl”—is generally a monovalent, branched or unbranched hydrocarbon group having 1 to 6 carbon atoms which can optionally be substituted with one or more halogen atoms—preferably fluorine—or one or more hydroxyl groups which can be identical or different among one another. Examples which may be mentioned are the following unsubstituted hydrocarbon groups:

methyl, ethyl, propyl, 1-methylethyl (iso-propyl), butyl, 1-methylpropyl, 2-methylpropyl, 1,1-dimethylethyl, n-pentyl, 1-methylbutyl, 2-methylbutyl, 3-methylbutyl, 1,1-dimethyl-propyl, 1,2-dimethylpropyl, 2,2-dimethylpropyl, 1-ethyl-propyl, hexyl, 1-methylpentyl, 2-methylpentyl, 3-methyl-pentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-tri-methylpropyl, 1,2,2-trimethylpropyl, 1-ethyl-1-methylpropyl and 1-ethyl-2-methylpropyl.

The same applies correspondingly to other—for example smaller or larger—alkyl groups, such as, e.g., C₁-C₃ alkyl or C₄-C₆ alkyl or similarly to higher alkyl groups which can be a monovalent branched or unbranched C₇-C₂₀ alkyl group which can optionally be substituted with one or more halogen atoms—preferably fluorine—which can be identical or different among one another. Examples which may be mentioned are the following preferred unsubstituted hydrocarbon groups: branched or unbranched heptyl, octyl, nonyl, decyl, undecyl, dodecyl, tetradecyl, hexadecyl, dodecadecyl and eicosyl.

Haloalkyl is the corresponding C₁-C₆ or higher alkyl groups which can be substituted with one or more halogen atoms—identically or differently independently of one another.

C₃-C₆ alkenyl is generally a branched or unbranched hydrocarbon radical having 3 to 6 carbon atoms having one, or optionally a plurality of, double bonds which can optionally be substituted with one or more halogen atoms—preferably fluorine—which can be identical or different among one another. Examples which may be mentioned are the following hydrocarbon groups:

2-propenyl (allyl), 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 3-methyl-4-pentenyl, 4-methyl-4-pentenyl, 1,1-dimethyl-2-butenyl, 1, crotyl-1-dimethyl-2-butenyl, 1,1-dimethyl-3-butenyl, 1,2-dimethyl-2-butenyl, 1,2-dimethyl-2-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl and 1-ethyl-2-methyl-2-propenyl.

C₃-C₆ alkynyl is generally a branched or unbranched hydrocarbon group having 3 to 6 carbon atoms having one, or optionally a plurality of, triple bonds which can optionally be substituted with one or more halogen atoms—preferably fluorine—which can be identical or different among one another. Examples which may be mentioned are the following hydrocarbon groups:

2-propynyl (propargyl), 2-butynyl, 3-butynyl, 1-methyl-2-propynyl, 2-methyl-2-propynyl, 2-pentynyl, 3-pentynyl, 4-pentynyl, 1-methyl-2-butynyl, 2-methyl-2-butynyl, 3-methyl-2-butynyl, 1-methyl-3-butynyl, 2-methyl-3-butynyl, 3-methyl-3-butynyl, 1,1-dimethyl-2-propynyl, 1,2-dimethyl-2-propynyl, 1-ethyl-2-propynyl, 2-hexynyl, 3-hexynyl, 4-hexynyl, 5-hexynyl, 1-methyl-2-pentynyl, 2-methyl-2-pentynyl, 3-methyl-2-pentynyl, 4-methyl-2-pentynyl, 1-methyl-3-pentynyl, 2-methyl-3-pentynyl, 3-methyl-3-pentynyl, 4-methyl-3-entynyl, 1-methyl-4-pentynyl, 3-methyl-4-pentynyl, 4-methyl-4-pentynyl, 1,1-dimethyl-2-butynyl, 1,1-dimethyl-2-butynyl, 1,1-dimethyl-3-butynyl, 1,2-dimethyl-2-butynyl, 1,2-dimethyl-3-butynyl, 1,3-dimethyl-2-butynyl, 1,3-dimethyl-3-butynyl, 2,2-dimethyl-3-butynyl, 2,3-dimethyl-2-butynyl, 2,3-dimethyl-3-butynyl, 1-ethyl-2-butynyl, 1-ethyl-3-butynyl, 2-ethyl-1-butynyl, 2-ethyl-2-butynyl, 2-ethyl-3-butynyl, 1,1,2-trimethyl-2-propynyl, 1-ethyl-1-methyl-2-propynyl and 1-ethyl-2-methyl-2-propynyl.

Alkylene is generally an unbranched divalent hydrocarbon group having 1 to 12 carbon atoms or a branched divalent hydrocarbon group having 3 to 6 carbon atoms such as, e.g., 2-methylpropylene, pentylene and the like.

The same applies correspondingly to the unsaturated C₃-C₆ alkenylene and C₃-C₆ alkynylene bridges.

Cycloalkyl is—unless defined otherwise—a saturated monovalent cyclic hydrocarbon group which can have three to eight carbon atoms and which can optionally be substituted with one or more halogen atoms—preferably fluorine—or one or more hydroxyl groups which can be identical or different among one another. Examples which may be mentioned are:

cyclopropyl, cyclohexyl, cycloheptyl or cyclooctyl. The carbon chain can be interrupted here by one or more heteroatoms—such as, e.g. nitrogen, oxygen or sulfur. Examples which may be mentioned are 1,4-dioxixane (dioxane), 2,5-dihydrofuran, tetrahydrofuran, γ-pyran, 2H-1,3-dioxole, tetrahydropyrrole, 2,5-dihydropyrrole, piperidine, piperazine, tetrahydrothiophene, morpholine, 1,2-oxathiolane, 1,3-thiazole, 1H-4,1,2-thiadiazine, 1,2-oxathiepane, γ-thiopyran, 1,4-thiazixine (1,4-thiazine).

Preferred examples of oxygen-containing heterocyclic and hydroxyl-substituted cyclic systems are, inter alia, for example the carbohydrates (pentoses and also hexoses) such as, e.g.: arabinoses, xyloses or riboses and glucoses, mannoses, galactoses, fructoses or sorboses.

Aryl is generally a monovalent monocyclic or bicyclic aromatic substituent having 6 to 10 ring atoms which optionally independently of one another with one or more, preferably with one or two, substituents selected from the group containing C₁-C₆ alkyl, C₁-C₆ haloalkyl, C₁-C₆ alkenyl, C₁-C₆ alkynyl, heteroalkyl, cycloalkyl, cycloalkylalkyl, halogen, cyano, nitro, acyloxy, amino, monoalkyl-substituted amine, dialkyl-substituted amine, acylamino, hydroxylamino, amidino, guanidino, —OR [where R hydrogen, C₁-C₆ alkyl, C₁-C₆ haloalkyl, C₃-C₆ alkenyl, C₃-C₆ alkynyl, cycloalkyl, cycloalkylalkyl], —S(O)nR [where can be an integer 0, 1 or 2 and R can be hydrogen, C₁-C₆ alkyl, C₁-C₆ haloalkyl, C₃-C₆ alkenyl, C₃-C₆ alkynyl, cycloalkyl, cycloalkylalkyl], —C(O)R [where R can be hydrogen, alkyl, alkenyl, cycloalkyl, C₁-C₆ haloalkyl], —COOR [where R can be hydrogen, C₁-C₆ alkyl, C₁-C₆ haloalkyl, C₃-C₆ alkenyl, C₃-C₆ alkynyl, cycloalkyl, C₁-C₆ haloalkyl], -(alkylene)COOR [where R can be hydrogen, alkyl, alkenyl, cycloalkyl, C₁-C₆ haloalkyl], —CONR′R″ or -(alkylene)CONR′R″ [where R′ and R″ independently of one another can be hydrogen, C₁-C₆ alkyl, C₁-C₆ haloalkyl, C₃-C₆ alkenyl, C₃-C₆ alkynyl, cycloalkyl, haloalkyl].

Aryl—unless stated otherwise—is also an aromatic mononuclear or polynuclear group having 4 to 22 carbon atoms which can contain one or two heteroatoms. Examples which may be mentioned are: naphthyl, anthracyl or pyrolyl, furanyl, thiophenyl, pyridinyl, pyridazinyl, pyrimidinyl or pyrazinyl.

Aralkyl, in the context of the above definition, is a mononuclear or polynuclear aryl group—as defined above—which is bonded to the hydrazine partial structure via a C₁-C₆ alkylene, C₃-C₆ alkenylene or a C₃-C₆ alkynylene bridge for which the definition of C₁-C₆ alkyl, C₃-C₆ alkenyl and C₃-C₆ alkynyl groups—as listed above—apply correspondingly. In the context of the present invention, the benzyl group is preferred.

Halogen—unless stated otherwise—is fluorine, chlorine, bromine or iodine, but preferably fluorine or chlorine.

The carrier surfaces derivatized according to the invention with hydrazine or with derivatives thereof may, depending on the chemical functionality of the groups situated on the surface, be converted to the desired products by analogy with methods already well known from the prior art.

First, the hydrazine derivatives required for the derivatization of the carrier may readily be prepared using standard methods known from the prior art.

In this manner, the reaction of hydrazine with alkyl halides [alkyl-X] (X=halogen) and sulfates (X=sulfate) usually delivers the unsymmetrical bis-substitution product [H₂N—N(alkyl)₃⁺X⁻ or H₂N—N(alkyl)₂].

In order, on the other hand, to obtain monoalkyl-hydrazines, for example benzaldazine can be used as starting material which can be converted to the corresponding monoalkyl salt in a possible alternative using an alkyl sulfate. After hydrolytic cleavage, the desired hydrazine derivative of the H₂N—NH(alkyl) type can readily be made accessible.

Symmetrically substituted hydrazine derivatives can likewise be synthesized in an uncomplicated manner with the aid of protecting groups. For instance, the corresponding symmetrically substituted dimethylhydrazine can be produced, e.g., via twofold acyl-protected hydrazine derivatives of the H(acyl)N—NH(acyl) type by reaction with dimethyl sulfate and subsequent acidic saponification.

In addition, the reduction of dialkylnitrosamines of the RₐR_{b}N—NO type can open up further simple access to the preparation of unsymmetrically substituted hydrazine derivatives.

All of the synthesis pathways shown above are—in addition to numerous others—well known from the prior art and embody general specialist knowledge [see, e.g.: R. C. Larock, Comprehensive Organic Transformations, VCH Publishers, Weinheim 1989].

The hydrazine derivatives produced in this manner may be reacted with a multiplicity of functional groups which the carrier can already comprise per se or which are generated in a separate reaction step on the carrier surface—or which are incorporated via the spacer.

Thus the reaction of carbonyl functions with suitably substituted hydrazine derivatives—as is likewise well known from the prior art—leads to the corresponding hydrazones which—if desired—can be further derivatized in a further step.

Furthermore, it is possible to react suitable carboxyl functions with hydrazine or with derivatives thereof—optionally in the presence of a coupling reagent—such as, e.g. EDC [N-ethyl-N′-(3-dimethylaminopropyl)carbodiimide hydrochloride], HBTU [2-(1H-benzotriazol-1-yl)-1,1,3,3-tetramethyluronium hexafluorophosphate] or TSTU [N,N,N′,N′-tetramethyl-O-(succinimidyl)uoronium tetrafluoroborate].

In addition, carboxyl groups activated from the start can be used on the carrier material—such as, e.g. acid halides or acid anhydrides or carbonyl imidazoles.

Further possibilities are the use of tresyl groups, azlactone- and pentafluorophenol-activated carriers. The use of activated carboxyl groups is preferred according to the invention.

In the context of the invention, epoxide groups are particularly preferred as functional groups for the reaction with hydrazine or with a hydrazine derivative.

In the reaction with hydrazine or with hydrazine derivatives, the epoxy ring opens, with formation of a hydroxyl function and also a covalent bond to the hydrazine partial structure.

The abovementioned functional groups can be partial structures of the linker—preferably if the spacer embodies only one covalent single bond or one double bond, or else they themselves embody partial structures of the spacer and are therefore only available after introduction of the spacer.

The present invention further relates to a method for purifying or isolating nucleic acids which comprises: providing the matrix modified according to the invention with hydrazine or with a hydrazine derivative, which matrix is to be used in the method. The subsequent immobilization step comprises combining the matrix with the mixture which contains the nucleic acid to be isolated in addition to at least one impurity at a first pH under conditions under which the desired nucleic acid is adsorbed to the matrix, separating the matrix containing the immobilized nucleic acid from the mixture and desorbing the nucleic acid at a desorption pH, wherein the desired nucleic acid(s) is (are) desorbed from the matrix.

Corresponding to the first part step of the above-described method—namely adsorbing the nucleic acid to the matrix modified with hydrazine and/or with one or more hydrazine derivatives—the complex which is formed from the nucleic acid with the partial structure of the hydrazine or the hydrazine derivatives embodies a further subject matter of the present invention.

In this case, the exact reaction conditions—which are necessary in order to ensure adsorption and desorption of the nucleic acids to and matrix—depend on various factors, including the properties of the matrix, the nature of the desired nucleic acid (DNA or RNA, molecular weight and nucleotide sequence composition, the pK_{b} and pKₐ of the basic, or where present, acidic partial structures, the density of the these structures on the surface and not least on the ability of the matrix thus modified to bind to the nucleic acid(s)).

In addition, it is not excluded that impurities in the mixture interfere adversely with the binding step in the adsorption of the nucleic acid(s).

The mixtures used here which contain the desired nucleic acids can either originate from synthetically produced reaction mixtures—as occur, e.g., in the PCR—or—as already mentioned at the outset—from biological samples.

Biological samples containing nucleic acids are taken to mean, in the context of the present invention, cell-free sample material, plasma, body fluids such as, for example, blood, serum, cells, leukocyte fractions, crusta phlogistica, sputum, urine, sperm, feces, smears, puncture samples, tissue samples of any type—such as, e.g. biopsies—tissue parts and organs, food samples which contain free or bound nucleic acids or nucleic acid-containing cells, environmental samples which contain free or bound nucleic acids or nucleic acid-containing cells—such as, e.g. organisms (unicellular or multicellular; insects etc.), plants and plant parts, bacteria, viruses, yeasts and other fungi, other eukaryotes and prokaryotes etc.

However, in the use of such samples, it is first necessary to destroy the cells in order to make the nucleic acids available for the adsorption step.

If the desired nucleic acid is RNA, the adsorption conditions must be set such that the adsorption of the desired RNA is promoted. If, in addition to the RNA, DNA is also present in the mixture, the adsorption conditions can be set such that preferably the binding of one species—in this case, in particular RNA—to the matrix is promoted. The specific adsorption conditions which are necessary, however, in order to make possible the adsorption and finally desorption of RNA, depends on the properties of the matrix and must be determined for each different type of matrix.

The matrix used according to the invention is preferably present in a form which can be separated from the mixture which contains the desired nucleic acid in addition to other substances/impurities by the action of an external force, after the mixture has been mixed with the matrix. For a person skilled in the art it is understandable that the type of the external force which is suitable for separating the matrix from the mixture is dependent on the form and the physical properties of the matrix. For example, in the simplest case, the separation can proceed under the action of gravity if the matrix is present, for example, in the form of the solid phase of a chromatographic separation system.

On the other hand, the matrix can be added—optionally batchwise—to the mixture of the nucleic acid and impurity (impurities) and subsequently by decanting or filtering be separated off again.

The external force which is used in the isolation method according to the invention can be a liquid which is at a high pressure, wherein the matrix forms the stationary phase of a high-pressure liquid chromatography.

Other types of external force, which are suitable for use in the method proposed according to the invention comprise vacuum filtration, centrifugation or preferably magnetic force.

In the use of magnetic force, preferably matrices which comprise permanently or temporarily magnetic material come into consideration. In the context of the present invention, preferably ferro- or ferrimagnetic particles are used, preferably selected from the group consisting of: γ-Fe₂O₃ (maghemite), Cr₂O₃, and also ferrites, in particular of the (M²⁺O)Fe₂O₃ type, wherein M²⁺ is a divalent transition metal cation, and preferably Fe₃O₄ (magnetite). Other ferro- or ferrimagnetic particles, however, can likewise be used. These particles have a median particle diameter of less than 5 μm, preferably of less than 1 μm, and particularly preferably in an interval between 0.0 and 0.8 μm, and very particularly preferably in a range from 7 to 300 nm.

Examples of suitable commercially available ferro- or ferrimagnetic particles are embodied by magnetic particles based on γ-Fe₂O₃— such as Bayoxide® E AB 21, based on ferrimagnetic magnetite as Bayoxide® E 8706, E 8707, E 8710 and E 8713H types (obtainable from Lanxess AG, Leverkusen, Federal Republic of Germany) and also as magnetic pigment 340 and as magnetic pigment 345 (obtainable from BASF AG, Ludwigshafen am Rhein, Federal Republic of Germany).

In addition, superparamagnetic materials can also be used. Superparamagnetic materials which come into consideration are Fe, Fe₃O₄, Fe₂O₃, the superparamagnetic ferrites Co, Ni and also binary and/or ternary compounds (alloys).

Mention may be made here by way of example of iron oxide crystals having a diameter of 300 angstroms and less.

The matrix according to the invention can be used in methods for purifying or isolating nucleic acids and which take place in conventional orders of magnitude and which are known, for example, from the prior art cited at the outset. The matrix according to the invention, however, is also suitable for use in or in combination with microfluidic systems as are adequately known from the prior art for nucleic acid purification and detection.

The examples hereinafter are intended to clarify the invention without restricting it:

### 1. Production of Hydrazine-Modified Beads

1.1 Direct Modification with Hydrazine

5 g of porous magnetic beads which are made of an iron oxide core enclosed by a sheath consisting of a polymethacrylate are suspended in 100 ml of deionized water in a 250 ml flask and admixed with 10 ml of hydrazine hydrate. The suspension is degassed by applying a water-jet vacuum twice and heated over a period of approximately 12 hours to a temperature of 70° C. with gentle stirring. The hydrazine-modified beads resulting from the reaction are transferred to a glass frit and in each case washed twice with demineralized water, with 10 mM hydrochloric acid and also with 100 mM sodium phosphate (pH 6.0) and demineralized water and subsequently suspended in 100 ml of demineralized water.

1.2. Indirect Modification with Hydrazine

5 g of porous magnetic beads which are made of an iron oxide core enclosed by a sheath consisting of a polymethacrylate are suspended in 100 ml of demineralized water in a 250 ml flask and admixed with 5 g of 6-aminohexanoic acid. The suspension is degassed by applying a water-jet vacuum twice and heated over a period of approximately 12 hours to a temperature of 70° C. with gentle stirring. The suspension is degassed by applying a water-jet vacuum twice and is heated to a temperature of 70° C. over a period of approximately 12 hours with gentle stirring. The beads modified with 6-aminohexanoic acid (6-aminocaproic acid) resulting from the reaction are transferred to a glass frit and washed in each case twice with demineralized water, with 10 mm hydrochloric acid and also with 100 mM sodium phosphate (pH 6.0) and demineralized water and subsequently suspended in 100 ml of demineralized water. Thereafter, 10 ml of hydrazine hydrate, 1 ml of a 100 mM solution of sodium phosphate in water (pH 6.0) containing 50 mg/ml of N-hydroxysuccinimide, 1 ml of a 100 mM solution of sodium phosphate in water (pH 6.0) containing 50 mg/ml of EDC are added. After addition is completed, the reaction mixture is stirred on a rotary evaporator for a period of 2 hours at room temperature (approximately 25° C.). The beads modified indirectly with hydrazine resulting from the reaction are transferred to a glass frit and washed in each case twice with demineralized water, with 10 mM hydrochloric acid and also with 100 mM sodium phosphate (pH 6.0) and demineralized water and subsequently suspended in 100 ml of demineralized water.

### 2. Biological Applications

### 2.1 DNA Purification Hydrazine-Modified Beads

10 μg of pUC21 (1 μg/μl) are added to each of the following buffer systems:

a) 390 μl of 100 M ammonium acetate (pH 5.5)
b) 390 μl of a buffer mixture of a Tris-containing EDTA buffer (pH 7.8-8.2), a sodium hydroxide-containing SDS solution (pH 13) and an acetic acid-containing potassium acetate buffer (pH 5.4-5.6) [buffer P1/P2/P3 from QIAGEN in Hilden (DE)], filtered,

P1: 50 mM Tris/Cl, pH 8.0; 10 mM EDTA

P2: 200 mM NaOH; 1% SDS

P3: 2 M K acetate pH 5.5.

After addition of 20 μl of a bead suspension (49 mg/ml), the mixture is incubated over a period of two minutes on a thermostated shaker at 1100 rpm. The beads are separated by means of magnetic force in the suspension and the supernatant is discarded. Thereafter, 300 μl of RNase-free water are added and the suspension is mixed over a period of one minute on a thermostated shaker at 1100 rpm. The beads are separated by means of magnetic force in the suspension and the supernatant is discarded. Thereafter, 100 μl of TRIS (2-amino-2-(hydroxymethyl)-1,3-propanediol) buffer (pH 9.5) are added and the suspension is mixed over a period of one minute on a thermostated shaker at 1100 rpm. Thereafter, the liquid phase (eluate) from the beads is separated by means of a commercially available magnetic separation device. This elution step is repeated and thereafter the DNA content is determined by determining the optical density (OD) and gel electrophoresis.

| Sample | | | | | |
|---|---|---|---|---|---|
| No. | Substance | OD 260 | 260/280 | 320 | μg DNA |
| 1 | RSH 001 1st eluate | 0.0420 | 1.6437 | 0.0073 | 0.35 |
| 2 | RSH 001 1st eluate | 0.0488 | 1.6185 | 0.0070 | 0.42 |
| 3 | RSH 001 2nd eluate | 0.1442 | 1.9549 | 0.1163 | 0.28 |
| 4 | RSH 001 2nd eluate | 0.0192 | 1.5809 | 0.0057 | 0.14 |
| Σ | | | | | 0.60 |
| 5 | RSH 002 1st eluate | 0.0276 | 1.6379 | 0.0073 | 0.20 |
| 6 | RSH 002 1st eluate | 0.0332 | 2.0450 | 0.0102 | 0.23 |
| 7 | RSH 002 2nd eluate | 0.0340 | 1.6273 | 0.0192 | 0.15 |
| 8 | RSH 002 2nd eluate | 0.0153 | 1.6281 | 0.0075 | 0.08 |
| Σ | | | | | 0.33 |
| 9 | RSH 003 1st eluate | 0.0681 | 1.7274 | 0.0136 | 0.55 |
| 10 | RSH 003 1st eluate | 0.0744 | 1.7390 | 0.0214 | 0.53 |
| 11 | RSH 003 2nd eluate | 0.0443 | 1.7028 | 0.0281 | 0.16 |
| 12 | RSH 003 2nd eluate | 0.0219 | 1.7216 | 0.0042 | 0.18 |
| Σ | | | | | 0.71 |
| 1 | RSH 001 1st eluate | 0.0990 | 2.6876 | 0.0145 | 0.85 |
| 2 | RSH 001 1st eluate | 0.1001 | 2.7022 | 0.0132 | 0.87 |
| 3 | RSH 001 2nd eluate | 0.0458 | 3.6169 | 0.0131 | 0.33 |
| 4 | RSH 001 2nd eluate | 0.0869 | 4.4566 | 0.0210 | 0.66 |
| Σ | | | | | 1.36 |
| 5 | RSH 002 1st eluate | 0.0828 | 2.6531 | 0.0178 | 0.65 |
| 6 | RSH 002 1st eluate | 0.1478 | 3.8327 | 0.0185 | 1.29 |
| 7 | RSH 002 2nd eluate | 0.0624 | 4.7887 | 0.0196 | 0.43 |
| 8 | RSH 002 2nd eluate | 0.0688 | 5.2687 | 0.0185 | 0.50 |
| Σ | | | | | 1.44 |
| 9 | RSH 003 1st eluate | 0.1639 | 3.7625 | 0.0336 | 1.30 |
| 10 | RSH 003 1st eluate | 0.2323 | 4.3711 | 0.0356 | 1.97 |
| 11 | RSH 003 2nd eluate | 0.0792 | 5.4754 | 0.0180 | 0.61 |
| 12 | RSH 003 2nd eluate | 0.0570 | 2.9812 | 0.0149 | 0.42 |
| Σ | | | | | 2.15 |
| 1 | RSH 001 1st eluate | 0.2371 | 2.1917 | 0.0813 | 1.56 |
| 2 | RSH 001 1st eluate | 0.1646 | 2.1971 | 0.0315 | 1.33 |
| 3 | RSH 001 2nd eluate | 0.1129 | 1.7942 | 0.0253 | 0.88 |
| 4 | RSH 001 2nd eluate | 0.0897 | 1.5896 | 0.0246 | 0.65 |
| Σ | | | | | 2.21 |
| 5 | RSH 002 1st eluate | 0.0534 | 3.0997 | 0.0077 | 0.46 |
| 6 | RSH 002 1st eluate | 0.0584 | 3.3729 | 0.0092 | 0.49 |
| 7 | RSH 002 2nd eluate | 0.0101 | 1.8410 | 0.0045 | 0.06 |
| 8 | RSH 002 2nd eluate | 0.0251 | 2.7947 | 0.0062 | 0.13 |
| Σ | | | | | 0.57 |
| 9 | RSH 003 1st eluate | 0.2453 | 2.1139 | 0.0409 | 2.04 |
| 10 | RSH 003 1st eluate | 0.1612 | 1.8893 | 0.0352 | 1.26 |
| 11 | RSH 003 2nd eluate | 0.0916 | 2.4295 | 0.0190 | 0.73 |
| 12 | RSH 003 2nd eluate | 0.0717 | 2.1919 | 0.0113 | 0.60 |
| Σ | | | | | 2.32 |
| 1 | RSH 001 1st eluate | 0.2361 | 1.6969 | 0.0287 | 2.07 |
| 2 | RSH 001 1st eluate | 0.2394 | 1.7228 | 0.0215 | 2.18 |
| 3 | RSH 001 2nd eluate | 0.0456 | 1.7574 | 0.0066 | 0.39 |
| 4 | RSH 001 2nd eluate | 0.0528 | 1.6575 | 0.0080 | 0.45 |
| Σ | | | | | 2.55 |
| 5 | RSH 002 1st eluate | 0.0448 | 1.7728 | 0.0019 | 0.43 |
| 6 | RSH 002 1st eluate | 0.0597 | 2.0174 | 0.0025 | 0.57 |
| 7 | RSH 002 2nd eluate | 0.0273 | 1.8611 | 0.0010 | 0.26 |
| 8 | RSH 002 2nd eluate | 0.0106 | 1.8458 | 0.0000 | 0.11 |
| Σ | | | | | 0.69 |
| 9 | RSH 003 1st eluate | 0.4201 | 1.7599 | 0.0198 | 4.00 |
| 10 | RSH 003 1st eluate | 0.4515 | 1.7748 | 0.0234 | 4.28 |
| 11 | RSH 003 2nd eluate | 0.0594 | 1.7749 | 0.0076 | 0.52 |
| 12 | RSH 003 2nd eluate | 0.0927 | 1.7582 | 0.0262 | 0.67 |
| Σ | | | | | 4.74 |

The present invention therefore relates to hydrazine-modified matrices according to the general formula I

carrier-linker-spacer-NR₃—NR₁R₂ (I),

where

the carrier surface of the carrier is formed of an inorganic or organic carrier material,

the linker is a functional group which is bound via a covalent bond to the carrier surface or to the carrier material and is bound either via the spacers or directly to the hydrazine partial structure,

the substituents R₁, R₂ and R₃ independently of one another can be hydrogen, C₁-C₆ alkyl, C₃-C₆ alkenyl, C₃-C₆ alkynyl, a higher alkyl group, a cycloalkyl group or aryl or aralkyl and, in the case of R₃, one also one further covalent bond.

In a preferred embodiment, the present invention relates to a matrix having a carrier surface which is formed from a polymeric inorganic or organic carrier material.

In a more preferred embodiment, the present invention relates to matrices having a carrier surface which is selected from the group comprising agaroses, polyacrylamides, celluloses, polyacrylates, polymethacrylates, polystyrenes, latex methacrylates and/or metal oxides.

In a further preferred embodiment, the present invention relates to matrices which have a carrier surface which is selected from the group polymethacrylate homopolymers and copolymers of polymethacrylate with styrenes, acrylates or further methacrylate derivatives, and also optionally with crosslinkers.

In a particularly preferred embodiment, the present invention relates to matrices which have a carrier surface which possess a crosslinker which is selected from the group divinylbenzene or ethylene glycol dimethacrylate.

In a further particularly preferred embodiment, the present invention relates to matrices which have a carrier surface which possesses a porous polymethacrylate homopolymer or copolymer.

In another preferred embodiment, the present invention relates to matrices, the matrix surface of which is made of silicon dioxide or silica, or comprises silicon dioxide or silica.

In a more preferred embodiment, the present invention relates to matrices, the carrier surface of which is present in the form of silica gel, solid glass or diatomaceous earth or as a mixture of these substances.

In a further preferred embodiment, the present invention relates to matrices having a linker which is a covalent bond or an up to six-membered alkylene bridge which can be optionally substituted by one or more halogen, hydroxyl, amino and thiol groups and which can be optionally interrupted by one or more further functional groups.

In a more preferred embodiment, the present invention relates to matrices having a linker, the functional groups of which are selected from the group —O—, —NH—, —S—, —C(═O)—, —C(═O)—C(═O)—, —C(═O)—O—, —C(═O)—C(═O)—O—, —C(═S)—O—, —C(═O)—S—, —C(═O)—N—, —NH—C(═O)—NH—.

In a preferred embodiment, the present invention relates to matrices having a spacer which is a single bond or double bond, an alkylene bridge containing 1 to 12 carbon atoms, which alkylene bridge can be optionally interrupted by one or more oxygen atoms and/or sulfur atoms and/or amino groups and optionally by one or more carbonyl or carboxyl groups.

In a more preferred embodiment, the present invention relates to matrices having a spacer, the carbonyl groups of which can be embodied by dialdehyde and polyaldehyde groups.

In a further more preferred embodiment, the present invention relates to matrices having a spacer, the carboxyl groups of which can be carboxyl, dicarboxyl or polycarboxyl groups.

In a further more preferred embodiment, the present invention relates to matrices having a spacer, the alkylene bridge of which comprises ethylene glycol diglycidyl ether, glycerol diglycidyl ether, glycerol triglycidyl ether ethylene glycol, polyethylene glycol, propylene glycol and/or substituted derivatives thereof.

In a preferred embodiment, the present invention relates to matrices having a hydrazine partial structure in which R₁, R₂ independently of one another are hydrogen and R₃ is a further covalent bond or hydrogen.

In a further embodiment, the present invention relates to a method for producing the matrices according to the invention in which a linker is generated on the carrier material or on the carrier material surface and optionally is reacted with a spacer or directly with hydrazine or a hydrazine derivative.

In a further embodiment, the present invention relates to the use of the matrices according to the invention for isolating biomolecules.

In a further embodiment, the present invention relates to a method for isolating biomolecules using the matrices according to the invention, which comprises the following steps:

(a) providing the matrix modified with hydrazine or with a hydrazine derivative;

(b) combining the matrix with a mixture containing the biomolecule to be isolated or preferably containing the nucleic acid to be isolated in addition to at least one impurity;

(c) incubating the matrix and the mixture at an adsorption pH, wherein the biomolecule or preferably the desired nucleic acid is immobilized on the matrix;

(d) separating the matrix containing the biomolecule or containing the immobilized nucleic acid(s) from the mixture;

(e) combining the matrix containing the biomolecule or containing the immobilized nucleic acid(s) with an elution solution at a desorption pH, wherein the desired biomolecule(s) or nucleic acid(s) is/are desorbed from the matrix.

In a preferred embodiment, the present invention relates to a method for isolating nucleic acid(s) using the matrices according to the invention.

In a further embodiment, the present invention relates to nucleic acids which are bound in the form of a complex to a hydrazine partial structure or the partial structure of a hydrazine derivative of the matrix according to the invention.

In another embodiment, the present invention relates to the use of the matrices according to the invention in microfluidic systems and to microfluidic systems which contain the matrix according to the invention.

1. A hydrazine-modified matrix according to the general formula I
carrier-linker-spacer-NR₃—NR₁R₂ (I),
where
the carrier surface is formed of a polymeric inorganic or organic carrier material,
the linker is a functional group which is bound via a covalent bond to the carrier surface or to the carrier material of the carrier and is bound either via spacers or directly to the hydrazine partial structure,
the substituents R₁, R₂ and R₃ independently of one another can be hydrogen, C₁-C₆ alkyl, C₃-C₆ alkenyl, C₃-C₆ alkynyl, a higher alkyl group, a cycloalkyl group or aryl or aralkyl and, in the case of R₃, one also one further covalent bond.

2. The matrix as claimed in claim 1, wherein the carrier is formed of a polymeric inorganic or organic carrier material.

3. The matrix as claimed in claim 2, wherein the carrier surface is selected from the group comprising agaroses, polyacrylamides, celluloses, polyacrylates, polymethacrylates, polystyrenes, latex methacrylates and/or metal oxides.

4. The matrix as claimed in claim 3, wherein the carrier surface is selected from the group polymethacrylate homopolymers and copolymers of polymethacrylate with styrenes, acrylates or further methacrylate derivatives, and also with crosslinkers.

5. The matrix as claimed in claim 4, wherein the crosslinker is selected from the group divinylbenzene or ethylene glycol dimethacrylate.

6. The matrix as claimed in claim 4, wherein the carrier surface is a porous polymethacrylate homopolymer or copolymer.

7. The matrix as claimed in claim 2, wherein the metal oxide is silicon dioxide or silica.

8. The matrix as claimed in claim 7, wherein the silicon dioxide is present in the form of silica gel, solid glass or diatomaceous earth or as a mixture of these substances.

9. The matrix as claimed in claim 1, wherein the linker is a covalent bond or an up to six-membered alkylene bridge which can be optionally substituted by one or more halogen, hydroxyl, amino and thiol groups and which can be optionally interrupted by one or more further functional groups.

10. The matrix as claimed in claim 9, wherein the functional groups are selected from the group —O—, —NH—, —S—, —C(═O)—, —C(═O)—C(═O)—, —C(═O)—O—, —C(═O)—C(═O)—O—, —C(═S)—O—, —C(═O)—S—, —C(═O)—N—, —NH—C(═O)—NH—.

11. The matrix as claimed in claim 1, wherein the spacer is a single bond or double bond, an alkylene bridge containing 1 to 12 carbon atoms, which alkylene bridge can be optionally interrupted by one or more oxygen atoms and/or sulfur atoms and/or amino groups and optionally by one or more carbonyl or carboxyl groups.

12. The matrix as claimed in claim 11, wherein the carbonyl groups can be dialdehyde and polyaldehyde groups.

13. The matrix as claimed in claim 11, wherein the carboxyl groups can be carboxyl, dicarboxyl or polycarboxyl groups.

14. The matrix as claimed in claim 11, wherein the alkylene bridge comprises ethylene glycol diglycidyl ether, glycerol diglycidyl ether, glycerol triglycidyl ether ethylene glycol, polyethylene glycol, propylene glycol and/or substituted derivatives thereof.

15. The matrix as claimed in claim 1, wherein R₁, R₂ independently of one another are hydrogen and R₃ is a further covalent bond or hydrogen.

16. A method for producing matrices as claimed in claim 1, wherein a linker is generated on the carrier material or on the carrier material surface and optionally is reacted with a spacer or directly with hydrazine or a hydrazine derivative.

17. The use of a modified matrix as claimed in claim 1 for isolating biomolecules.

18. A method for isolating biomolecules using a matrix modified with hydrazine or with a hydrazine derivative as claimed in claim 1, comprising the following steps:
(a) providing the matrix modified with hydrazine or with a hydrazine derivative;
(b) combining the matrix with a mixture containing the biomolecule to be isolated in addition to at least one impurity;
(c) incubating the matrix and the mixture at an adsorption pH, wherein the biomolecule is immobilized on the matrix;
(d) separating the matrix containing the biomolecule from the mixture;
(e) combining the matrix containing the biomolecule with an elution solution at a desorption pH, wherein the desired biomolecule(s) is/are desorbed from the matrix.

19. The method as claimed in claim 16, wherein the biomolecule is one or more nucleic acids.

20. A nucleic acid, wherein it is bound in the form of a complex to a hydrazine partial structure or the partial structure of a hydrazine derivative of a matrix as claimed in claim 1.

21. A microfluidic system containing a matrix as claimed in claim 1.
